(19) United States
(12) Patent Application Publication
(10) Pub. No.: US 2022/0154146 A1
(43) Pub. Date: May 19, 2022

(54) METHOD OF IMPROVING THE IN VIVO SURVIVAL OF MESENCHYMAL STEM CELLS

(71) Applicant: The Administrators of the Tulane Educational Fund, New Orleans, LA (US)

(72) Inventors: Kim C. O'CONNOR, New Orleans, LA (US); Sean D. MADSEN, Seattle, WA (US); Bruce A. BUNNELL, Mandeville, LA (US)

(21) Appl. No.: 17/440,654

(22) PCT Filed: Mar. 18, 2020
(86) PCT No.: PCT/US2020/023459

§ 371 Date: Sep. 17, 2021

Related U.S. Application Data
(60) Provisional application No. 62/820,367, filed on Mar. 19, 2019.

(51) Int. Cl. C12N 5/0775; A61K 35/28

(52) U.S. Cl. CPC C12N 5/0663; C12N 2533/18; A61K 35/28

(57) ABSTRACT

Methods of improving the in vivo survival of mesencymal stem cells are described. The method comprising the steps of: a) selecting, from a heterogeneous group of MSCs, MSCs having high expression of NG2; b) expanding the MSCs selected in step a); c) attaching the MSCs expanded in step b) to a scaffold; and d) implanting the scaffold with the MSCs in a mammal.

### PRIOR RELATED APPLICATIONS

This invention claims priority to U.S. 62/820,367, filed on Mar. 19, 2019 and incorporated by reference in its entirety herein for all purposes.

### FEDERALLY SPONSORED RESEARCH STATEMENT

This invention was made, in part, with support provided by the United States government under Grant Nos. CBET-1066167 and CBET-1604129 awarded by the National Science Foundation. The Government has certain rights in this invention.

### FIELD OF THE DISCLOSURE

The present invention relates to novel methods to improve the survival of mesenchymal stem cells in vivo. These methods can be used to improve procedures and implants used in a variety of diseases where promotion of tissue repair is necessary for recovery or cure from disease.

### BACKGROUND OF THE DISCLOSURE

Mesenchymal stem cells (MSCs) possess a broad spectrum of regenerative properties, which are being deployed in clinical trials to treat numerous disorders. MSC applications range from repairing articular cartilage defects to improving neurological function after a stroke. The success of MSC therapies is dependent upon the survival of implanted stem cells. Engraftment applications rely on MSCs to integrate and replace damaged or diseased tissue, while non-engraftment applications leverage the continued presence of MSCs to secrete bioactive factors that promote tissue repair. Accordingly, standardization of MSC survival in vivo is essential to achieve consistent treatment outcomes.

Transplanted MSCs are stressed in vivo by a variety of factors, including ischemia at the implant site. In this harsh environment, MSC implants survive in vivo for only days to weeks, whereas, repair of tissues like bone takes months. To illustrate, the amount of rat MSCs in allografts decreased over 70% after 3 days in vivo according to one study but experienced only a 50% loss after 35 days according to another report. As a result, MSC implants are viable for only a fraction of the healing time.

Previous attempts to improve MSC survival in vivo include preconditioning the stem cells prior to implantation with growth factors and hypoxia. Other strategies to retain viable MSC implants have focused on manipulating the concentration and attachment of bioactive molecules in the stem cell microenvironment.

The literature is silent on the contribution of cellular heterogeneity to the survival of MSC implants. MSC cultures are a heterogeneous mixture of progenitors with different regenerative potentials at different stages of cellular aging. Long-term culture of MSCs revealed continuous and incremental changes to their global gene expression profile towards a senescent phenotype, as cellular aging is a result of accumulated DNA damage from replicative stress and can result in a functional change that is detrimental to the regenerative properties of MSCs, including a decrease in proliferation potential. Although stem cell aging is being studied extensively in vitro, to date, there has been no work to investigate the in vivo survival of aging MSCs of any kind. This is a critical knowledge gap in light of the importance of cell survival to MSC therapies and the impaired proliferation potential of aging MSCs.

TRAIL receptor CD264 has been reported as the first known surface marker of cellular aging for MSCs. CD264 is upregulated concomitantly with p21 at an intermediate stage of cellular aging and remains upregulated through senescence. It is reported that MSC cultures from young donors contained 20-40% CD264⁺ cells, with even higher CD264⁺ cell content possible for older donor cultures. In addition, a strong inverse correlation of CD264⁺ cell content in MSC cultures with their in vitro proliferation and differentiation potential has also been reported.

On the other hand, while it has been reported that the level of NG2 expression positively correlates with the prolifieration and trilineage potential of MSCs in vitro, such correlation was never extended to in vivo MSC survival. as discussed above the in vivo conditions are different from in vitro conditions, and the stress response of cells to unfavorable environment activates different pathways to cope with potentially lethal stimuli.

Therefore, there is still the need for better screening method for in vivo MSC survival in order to improve the efficacy of MSC therapies.

### SUMMARY OF THE DISCLOSURE

A method of improving mesenchymal stem cells (MSCs) in vivo survival is described herein. After collecting MSCs from a subject, the first step is to select and isolate the MSCs with high expression of neuron glial antigen 2 (NG2^{Hi}), as these MSCs are found to have the longest in vivo survival rate. The NG2^{Hi} MSCs are then expanded to necessary amount for MSC therapy. Once the number of MSCs is sufficient, depending on the different treatment methods, a scaffold may be provided onto which the MSCs can attach. The resulting scaffold with attached MSCs can then be implanted into a patient.

A composition of MSCs having high in vivo survival rate is also described herein. In the composition, at least 50% of MSCs have high NG2 expression. The composition can also comprise less than 30% of CD264⁺ MSCs or more than 40% of CD264⁺ MSCs. In embodiment, the composition comprises less than 15% CD264⁺ MSCs, or less than 10% CD264⁺ MSCs. In embodiments, the composition comprises more than 50% CD264⁺ MSCs.

In embodiments, the NG2^{Hi} MSCs are selected and isolated by flow cytometry, in which only the cells in the top 30% expression level are selected. In one embodiment, only cells in the top 15% expression level are selected. In one embodiment, only cells in the top 10% expression level are selected.

In embodiments, during the expansion step, CD264⁺ cells can be removed, such that only less than 30% of the expanded MSCs are CD264⁺. In one embodiment, only less than 15% of the expanded MSCs are CD264⁺. In one embodiment, only less than 10% of the expanded MSCs are CD264⁺.

In embodiments, during the expansion step, CD264⁺ cells can be enriched, such that at least 40% of the expanded MSCs are CD264⁺. In one embodiment, at least 50% of the expanded MSCs are CD264⁺.

In embodiments, after the desired therapeutic effect has been reached, or after a predetermined period of time, the CD264⁺ MSCs are removed or killed from the scaffold.

A method of implanting mesenchymal stem cells in a mammal is described, comprising the steps of: a) selecting and/or isolating, from a heterogeneous group of MSCs, MSCs having top 30% expression level of neuron-glial antigen 2 (NG2); b) expanding the MSCs selected in step a), wherein the expanded MSCs having CD264⁺ are removed or killed; c) attaching the MSCs expanded in step b) to a scaffold; and d) implanting the scaffold with the MSCs in a mammal.

In embodiments, after the removal of CD264⁺ MSCs, only 15% or less of the expanded MSCs are CD264⁺. In embodiments, after the removal of CD264⁺ MSCs, only 10% or less of the expanded MSCs are CD264⁺.

A method of implanting mesenchymal stem cells in a mammal is described, comprising the steps of: a) selecting and/or isolating, from a heterogeneous group of MSCs, MSCs having top 30% expression level of neuron-glial antigen 2 (NG2); b) expanding the MSCs selected in step a), wherein the expanded MSCs having CD264⁺ are enriched; c) attaching the MSCs expanded in step b) to a scaffold; and d) implanting the scaffold with the MSCs in a mammal.

In embodiments, after the enrichment of CD264⁺ MSCs, at least 40% the expanded MSCs are CD264⁺. In embodiments, after the enrichment of CD264⁺ MSCs, at least 50% of the expanded MSCs are CD264⁺.

A method of making a scaffold having mesenchymal stem cells (MSCs) attached thereon is also described, comprising the steps of: a) selecting and/or isolating, from a heterogeneous group of MSCs, MSCs that do not express CD264; b) expanding the MSCs selected in step a), wherein the expanded MSCs having the bottom 50% expression level of NG2 are removed; and c) attaching the MSCs expanded in step b) to a scaffold.

In embodiments, in the expansion step b), the MSCs having the bottom 70% expression level of NG2 are removed.

As used herein, “scaffold” means a three dimensional structure that serves as a suitable support for the grown and proliferation of the stem cells, does not interfere with stem cell growth and viability, and permits adherence of the human mesenchymal stem cells. In embodiments, the scaffold is tricalcium phosphate/hydroxyapatite (HA/TCP) scaffold. In embodiments, the scaffold can be an elastomeric matrix that is preferably porous, and is reticulated and resiliently-compressible. For example, the elastomeric matrix can be made from a thermoplastic elastomer such as polycarbonate polyurethanes, polyether polyurethanes, polysiloxane polyurethanes, hydrocarbon polyurethanes, polyurethanes with mixed soft segments, and mixtures thereof, and preferably is made from polycarbonate polyurethane. It is also within the confines of the present disclosure that the matrix can be coated with a coating material such as collagen, fibronectin, elastin, hyaluronic acid or mixtures thereof to facilitate cellular ingrowth and proliferation.

As used herein, “high expression” refers to a expression level that is higher than the average expression level in any given group of heterogeneous cells. In one embodiment, “high expression” refers to the top 30% expression level among a group of cells. In one embodiment, “high expression” refers to the top 15% expression level among a group of cells. In one embodiment, “high expression” refers to the top 10% expression level among a group of cells. Genetically overexpressed NG2 can also lead to high expression of NG2 protein.

As used herein, “expression level” refers to the level of expression that can be used to sort the cells. There are several methods to measure the expression level of a surface marker, such as using the kinetics of antibody binding and radioactively labeled ligands, as well as using calibrated beads and flow cytometry, as known in the art. In embodiments, the expression level of NG2 or CD264 is measured using flow cytometry, and cells are sorted accordingly.

“Overexpression” or “overexpressed” is defined herein to be at least 150% of protein activity as compared with an appropriate control species or as having detectable expression of a gene not normally present in that host. Overexpression can be achieved by mutating the protein to produce a more active form or a form that is resistant to inhibition, by removing inhibitors, or adding activators, and the like. Overexpression can also be achieved by removing repressors, adding multiple copies of the gene to the cell, using highly active expression vectors, or upregulating the endogenous gene, and the like. An overexpressed gene can be represented by the ⁺ symbol, e.g., CD264⁺. In contrast, “expression” refers to normal levels of activity or better.

The use of the word “a” or “an” when used in conjunction with the term “comprising” in the claims or the specification means one or more than one, unless the context dictates otherwise.

The term “about” means the stated value plus or minus the margin of error of measurement or plus or minus 10% if no method of measurement is indicated.

The use of the term “or” in the claims is used to mean “and/or” unless explicitly indicated to refer to alternatives only or if the alternatives are mutually exclusive.

The terms “comprise”, “have”, “include” and “contain” (and their variants) are open-ended linking verbs and allow the addition of other elements when used in a claim.

The phrase “consisting of” is closed, and excludes all additional elements.

The phrase “consisting essentially of” excludes additional material elements, but allows the inclusions of non-material elements that do not substantially change the nature of the invention.

The following abbreviations are used herein:

| ABBREVIATION | TERM |
|---|---|
| GFP | Green fluorescent protein |
| HA/TCP | Hydroxyapatite/tricalcium phosphate |
| hBM-MSC | Human bone marrow mesenchymal stem cell |
| MFI | Mean fluorescent intensity |
| NG2 | Neuron-glial antigen 2 |

### BRIEF DESCRIPTION OF THE DRAWINGS

FIG. 1 shows an overview of project design to quantify the in vivo survival of aging CD264⁺ MSCs. Following lentiviral transduction, MSCs were evaluated for transgene expression and stem cell fitness. GFP-FLuc MSCs were amplified and sorted into CD264⁺ and control CD264⁻ populations. Quality control assessment of CD264-sorted cells evaluated sort purity, aging phenotype and single-cell survival. Attachment of CD264⁺ and CD264⁻ GFP-Fluc MSCs to HA/TCP porous scaffolds was quantified, and then seeded scaffolds were aggregated with mouse thrombin and fibrinogen. Aggregated MSC constructs (both CD264⁺ and CD264⁻) were implanted subcutaneously on the dorsal surface of immunodeficient mice, and bioluminescent imaging was performed every 3-4 days for 31 days. Bioluminescent signal was used to determine the survival kinetics of CD264-sorted MSCs. On the final day of imaging, implants were harvested for histological analysis.

FIG. 2 shows in vitro comparison of sorted MSCs and aging CD264 phenotype, in terms of colony forming efficiency, morphology and senescence. (a) Colony-forming efficiency was evaluated in 10 cm tissue culture plates using crystal violet staining for CD264⁺ and CD264⁻ MSCs from each sort (n=6 biological replicates per group). Median values are depicted as bars. ((b), (c)) Select CD264 sorts from both MSC donors were evaluated for SA β-Gal activity at pH 6.0. Data are reported as mean±SEM (n=3 biological replicates). Scale bars: 100 μm. *p<0.05, **p<0.01, and ***p<0.0001 vs donor-matched CD264⁻ MSCs.

FIG. 3 shows in vitro survival of CD264+ and CD264− MSCs. (a-c) single-cell survival of CD264⁺ and CD264⁻ MSCs. CD264 sorted cells were inoculated at one cell/well by limiting dilution. (a) cell survival on day 3, (b) cell survival on day 7, and (c) colonies formed (≥10 cells) from surviving cells on day 7. Data are expressed as the mean±SEM for n=3 biological replicates (30-40 single cells/replicate). *p<0.05 versus donor-matched CD264⁻ MSCs. (d) CD264-sorted GFP-FLuc MSCs from Donor 1 were seeded on 40 mg HA/TCP granules, aggregated with mouse thrombin and fibrinogen, and cultured for 2 months. (d) Temporal profile of background-corrected bioluminescent signals after 2 months in vitro.

FIG. 4 shows MSC attachment to HA/TCP granules and scaffold aggregation. (a)-(c) Fluorescent images of transduced MSCs that were cultured on 40 mg porous HA/TCP granules for 6 hours at the stated inoculum. Scale bars=200 μm. Arrows indicate diameter of inner pore (125 μm, a) and outer shell (500 μm, b). (d)-(f) Attachment of 10⁶ CD264⁺ (triangle) and CD264⁻ (circle) MSCs from donor 1 (d) and donor 2 (e) evaluated by cells remaining in suspension (main, mean±SEM, n=4 biological replicates), and DNA content of attached cells after 6 hours (f) (inset, mean±SEM, n=3 biological replicates). (g, h) Scaffold architecture before and after aggregation with mouse thrombin and fibrinogen. Scale bars=1 cm.

FIG. 5 shows in vivo survival of CD264-sorted MSCs. Mice were implanted with 10⁶ CD264⁺ and CD264⁻ GFP-FLuc MSCs/40 mg HA/TCP scaffold, and images of the bioluminescent signal radiating from the mice were acquired every 3-4 days for a period of 31 days. (a) Image sequences of representative mice from both MSC donors implanted with CD264-sorted MSCs. The number in the upper right corner of image columns indicates the number of days post-implantation. (b, c) Background-corrected bioluminescent signal from CD264⁺ and CD264⁻ implants that corresponds to the representative image sequences. (d-f) Signal half-life of the bioluminescence from CD264⁺ and CD264⁻ MSCs for each donor (n=6 biological replicates per group). Mouse gender is denoted by triangle (male) and circle (female). (d-f) In vivo half-life of CD264-sorted hBM-MSCs and correlations with colony-forming efficiency. Mean values depicted as bars. Linear regression lines and Pearson's correlation coefficients are presented on each bivariate graph. **p<0.01 versus Donor 1. 1, Donor 1; 2, Donor 2; hBM-MSCs, human bone marrow mesenchymal stem cells.

FIG. 6 shows mean fluorescence intensity ratio of NG2 expression on hBM-MSCs (mean±SEM, n=3 biological replicates) from flow cytometric analysis of NG2 surface expression for both donor 1 and donor 2). The NG2 MFI ratio for donor 2 hBM-MSCs was on average >1.5 times the value for donor 1 hBM-MSCs. *p<0.05 vs donor 1. Abbreviations: APC: allophycocyanin; hBM-MSCs: human bone marrow mesenchymal stem cells; MFI: mean fluorescence intensity; NG2: neuron-glial antigen 2; SEM: standard error of the mean.

FIG. 7 shows eGFP ROI fraction of CD264⁺ and CD264⁻ implants from each hBM-MSC donor in male and female mice. Mean values depicted as bars. *p<0.05 versus Donor 1. 1, Donor 1; 2, Donor 2.

FIG. 8 shows that the half-lives of the sorted MSCs in this disclosure compare favorably to published MSC survival results. These studies use a variety of MSC sources, implant materials, and survival models resulting in a broad range of mean half-lives from 2-35 days. MSCs that were injected intravenously had the lowest half-life of any of the listed studies (<2 days, Vilalta et al., 2008). Studies that injected MSCs into specific tissue such as muscle or a calvarial defect lasted 3-4 times longer (6-8 days, Vilalta et al., 2008; Freitas et al., 2017 compared to the intravenous administration. Our survival results present significant improvements over similar ectopic survival assays using bone marrow-derived MSCs attached to ceramic scaffolds (Giannoni et al., 2010; Zimmerman et al., 2011; Manassero et al., 2016). It is notable that the ceramic construct with the shortest attachment period resulted in the lowest half-life of all scaffold-containing implants (Giannoni et al., 2010). Additionally, MSC retention is improved with the addition of matrix components, such as fibrinogen, to the microenvironment (Karoubi et al., 2009; Gianonni et al., 2010). Consistent with findings from Manassero et al. (2016), mean half-lifes in our study were comparable to osteochondral defect implantations sites and are among the highest values in the literature.

FIG. 9 shows the mRNA expression of osteogenesis marker collagen 1A1 and eGFP in ectopic implants of eGFP+ MSCs in NIH III mice. Fold change in expression measured with qPCR relative to mouse GAPDH. Implants excised one month after surgery. Dashed trendline. (rs>0.95, n=6).

### DETAILED DESCRIPTION

In the invention disclosed herein, a novel method of selecting MSCs capable of long in vivo survival and preparing an implant by attaching the MSCs on a scaffold, with optionally removing CD264+ cells in order to improve the therapeutic efficacy. These changes result in MSC half-life that is among the longest reported in the literature. The method of this disclosure is based on the surprise findings that (1) NG2 expression level is higher on the longer surviving MSCs in vivo, (2) CD264+, while a marker for senescent MSCs, does not negatively impact MSC survival in vivo, and (3) CD264+ cells are less therapeutically effective.

Prior to this disclosure, colony forming efficiency in cell culture is accepted as a measure of in vitro cell survival, and extrapolated as an indicator of in vivo cell survival. However, the inventors discovered that CD264+ and CD264− MSCs have comparable in vivo survival kinetics. Such results prompted the need for a new method of screening for long in vivo survival MSCs in order to achieve better MSC therapies.

Inventors also discovered that NG2 expression level is elevated in those MSCs that survived longer in vivo, comparing to the MSCs that have shorter halflives. Further, while CD264+ MSCs exhibit similar in vivo survival results, their senescent status still make them less desirable for MSC therapies, and therefore negative selection of CD264+ cells would result in a group of MSCs that have both longer half-lives and better therapeutic efficacy.

For example, CD264 may be upregulated in aging hBM-MSCs as a potential stress response to facilitate cell survival. The upregulation of CD264 has been noted in several stress responses including ischemic preconditioning, oxidative stress, and inflammatory signaling. Previous work suggests that CD264 expression may have a prosurvival effect on cancer cells by mediating antiapoptotic signaling. In this context, CD264 may function to counteract the replicative stress of cellular aging in hBM-MSCs by promoting survival, as evidenced by the persistence of these cells following implantation.

The efficiency of the MSC attachment to the scaffold and the resultant in vivo retention of cells hold promise to develop reliable therapeutics. The Mastergraft product was chosen as a cellular scaffold due to its numerous clinical applications including spinal fusion, iliac crest backfilling, and dental surgery. Adapting the Mastergraft Mini Granules to these specific methods could generate clinic-ready bone grafts with improved chances for successful implantation. Additionally, this method can be extended to other implant materials and geometries to improve in vivo MSC retention. Graft materials in a block format have been explored in vivo and coral-based bone grafts are clinically used biocompatible scaffolds that exhibit similar properties to the Mastergraft granules. Using the attachment and preparation method detailed in this study to produce MSC-based constructs result in improved in vivo MSC survival for varying scaffold types compared to previous studies.

Furthermore, negative selection with CD264 to standardize MSC composition for implantation could produce more efficacious therapies. Previous work has shown that CD264⁺ MSCs are present in all MSC cultures, and this disclosure demonstrates that this aging population of cells has robust in vivo survival. It is well-established that aging and senescent MSCs have weakened regenerative potential in vitro and late-passage MSCs have been shown to be less effective in the treatment of graft-versus-host disease in humans compared to early-passage cells. Due to their in vivo persistence and poor regenerative properties, it is necessary to remove the aging CD264⁺ MSCs from the heterogenous culture using negative selection. The resulting MSC cultures should have robust regenerative properties resulting in improved therapeutic outcomes when implanted.

Negative selection to remove CD264⁺ MSCs may not always be a viable option due to a donor's high CD264⁺ content. Enrichment of these aging MSCs through positive selection could provide alternate treatment strategies. For example, the prolonged in vivo survival of aging CD264⁺ MSCs can be used to exploit effects of the senescence-associated secretory phenotype (SASP). The SASP is a result of cellular reprogramming during senescence where the bioactive molecules secreted by the cell drastically change. There is extensive literature detailing the potential therapeutic applications of the SASP for the treatment of liver fibrosis, wound healing, immune cell recruitment, and tissue regeneration. Creating an implantable construct containing aging CD264⁺ MSCs with predictable in vivo survival will be a reliable method to consistently deliver the beneficial SASP for a targeted application. Once the desired effect is achieved, the implant could be physically removed or the aging CD264⁺ MSCs targeted with a senolytic drug to clear the senescent cells. Additionally, positive selection using CD264 allows rejuvenation of the aging MSCs to restore their regenerative properties. If the desired outcome is integration of autologous MSCs into the target tissue, rejuvenating the aging CD264⁺ MSCs, such as through transient p38 inhibition or p53 inactivation, would be a necessary step to achieve an efficacious graft at the proper therapeutic dosage.

Detailed descriptions of one or more preferred embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in any appropriate manner.

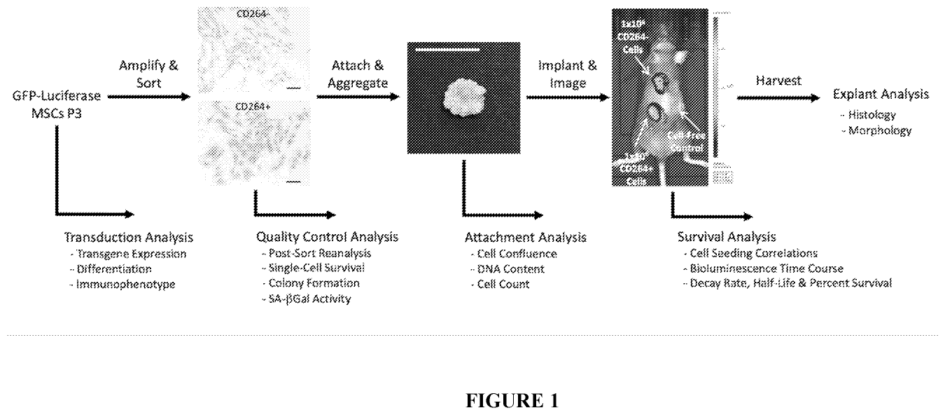

This disclosure employed a well-established in vivo survival model that monitors bioluminescence from subcutaneous implants of GFP-FLuc MSCs on the dorsum of immunodeficient mice (FIG. 1). Bone marrow MSC cultures in this study were obtained from donors, whose chronological age was matched to within one year (donor 1: 36 years old, donor 2: 37 years old). Our transduced cells conformed to the MSC criteria established by the International Society for Cellular Therapy, in which they retained plastic adherence, an MSC immunophenotype and robust trilineage differentiation (data not shown). Following differentiation, transduced MSCs maintained their bioluminescence and fluorescence (data not shown) to enable evaluation of their in vivo survival. MSCs in this study were amplified to passage 5 (P5), which is within the range of passage numbers for MSCs in clinical trials.

The in vivo results are also compared with in vitro results, particularly regarding the effect of NG2 expression level and CD264 expression on MSC survival. The comparison indicates that MSCs with high NG2 expression level tend to survive longer both in vitro and in vivo, whereas CD264⁺ and CD264⁻ MSCs do not show significant difference.

Aging Phenotype of CD264-Sorted Populations

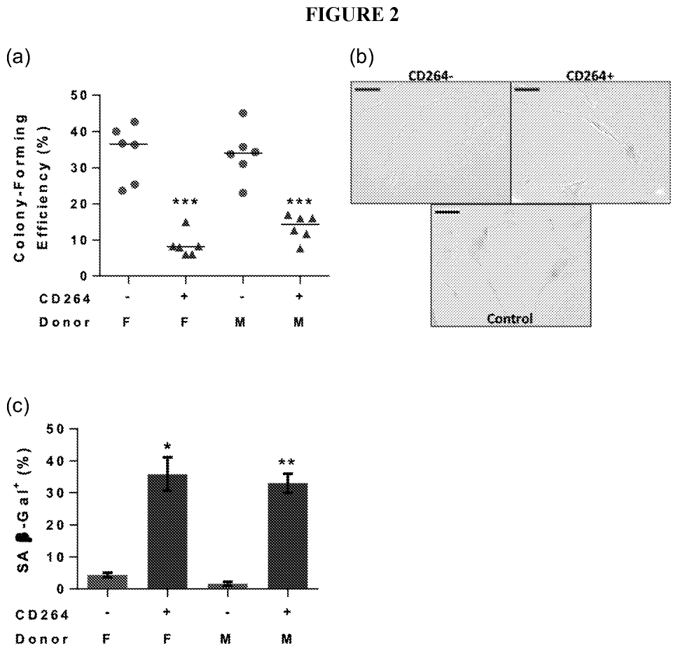

Cellular aging of MSCs was detected by expression of CD264. Heterogeneous cultures of transduced MSCs were 35% positive for CD264 expression based on a 1% isotype cutoff, consistent with the content of CD264⁺ cells that were observed for robust MSC cultures. P5 MSCs were sorted into aging CD264⁺ and control CD264⁻ populations immediately prior to implantation to avoid artifacts in survival from differences in expansion and sorting conditions. Post-sort reanalysis confirmed distinct fluorescent separation between CD264⁺ and CD264⁻ MSCs (data not shown). The colony-forming efficiency of each sorted population to be implanted was measured (FIG. 2(a)). Control CD264⁻ cells formed colonies with a median efficiency between 30-40% for both donors (FIG. 2(a)), a value that typifies early-passage MSC cultures. Colony-forming efficiency of CD264⁺ MSCs was 2.5-4.0 times less than their CD264⁻ counterparts, indicative of a loss of proliferation potential with cellular aging. Relative to the CD264⁻ control, CD264⁺ MSCs had an enlarged size that is emblematic of an aging morphology (FIG. 2(b)). Select batches of sorted cells were assayed for senescence-associated β-Gal activity, which was elevated in CD264⁺ MSCs and negligible for CD264⁻ MSCs (p<0.05 for donor 1, p<0.01 for donor 2, n=3, FIG. 2(c)).

In Vitro Single-Cell Survival

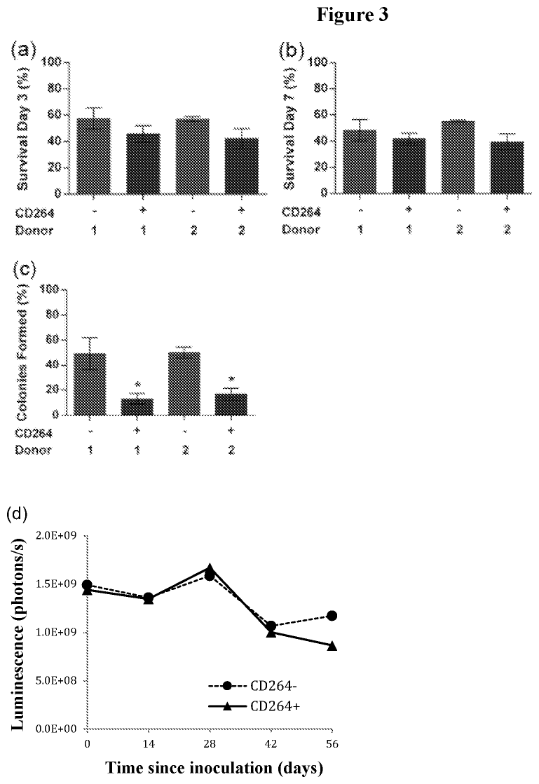

Single-cell survival was comparable between CD264⁺ and CD264⁻ MSCs when assessed by limiting dilution into 96-well plates for 7 days (FIG. 3). For each sort group, the inoculum had high viability >90% and a single-cell plating efficiency >30%. In total, 90-110 single-cell wells were analyzed per sort group for each donor. The percentage of single cells that survived on day 3 and day 7 was similar for matched CD264⁺ and CD264⁻ MSCs: ˜50% on average (FIG. 3(a-b)). No significant donor variation in single-cell survival was detected.

Nearly half of the single CD264⁻ cells that survived on day 7 formed colonies ≥10 cells. Surviving CD264⁺ MSCs formed colonies less efficiency at ˜15% (p<0.05, n=3 replicates, 30-40 single cells/replicate, FIG. 3(c)). FIG. 3 indicates that aging CD264⁺ MSCs have similar in vitro single-cell survival to control CD264⁻ MSCs, but they form colonies less efficiently due to compromised cell proliferation.

Long term in vitro survival was also observed for 2 months (FIG. 3(d)). For CD264-sorted GFP-Fluc MSCs seeded on 40 mg HA/TCP granules aggregated with mouse thrombin and fibrinogen, cultured for 2 months, FIG. 3(d) shows the temporal profile of background-corrected bioluminescent signals after 2 months in vitro. Taking the bioluminescence as an indirect indicator of MSC survival, CD264⁻ and CD264⁺ cells again show comparable in vitro survival results. FIG. 3(d) shows during the first 28 days the in vitro cells from Donor 1 remain approximately the same. Contrast this with FIG. 5(c), where the number of in vivo cells from the same donor steadily decreases from day 1 to 28. The comparison clearly shows that in vitro survival results cannot be directly translated into in vivo survival.

Cell Attachment to Scaffold

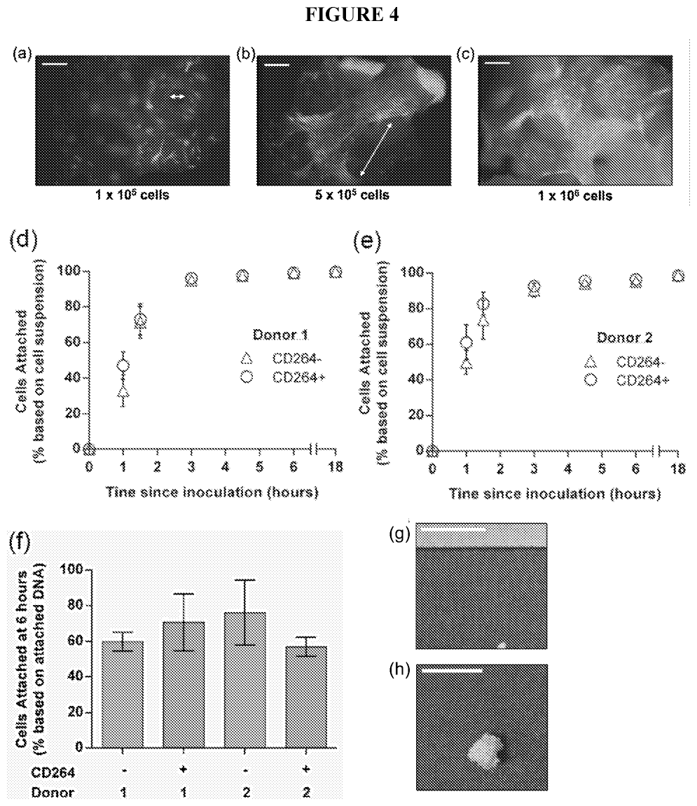

Before implanting the cells into mice, MSCs were first attached to medical-grade HA/TCP granules (0.5 mm-1.6 mm particle diameter), a porous scaffold that is frequently used for ectopic MSC implants. Fluorescence from the transduced MSCs revealed the scaffold architecture of interconnected hollow shells with an outer shell diameter of 500 μm and inner pore diameter of 125 μm connecting the shells (FIGS. 4(a-c)). Initially, the scaffold was inoculated at different seeding densities to establish conditions of confluency (FIGS. 4(a-c)). The scaffold became confluent at 1×10⁶ MSCs/40 mg granules (FIG. 4(c)). This seeding density was found previously to be optimal for MSC survival and was selected for these implants.

At this seeding density, all MSC preparations attached efficiently to the scaffold (FIG. 4(d)). Cell attachment to the HA/TCP granules was quantified over 18 h of mixing. It is estimated that >95% of MSCs from both donors attached to the granules after 6 h based on cells remaining in solution (FIG. 4(d-e)). This method can overestimate attachment from cells that have settled on the scaffold but not yet attached. In addition, it is estimated that cell attachment by measuring DNA content on the scaffold, which can underestimate attachment due to incomplete cell lysing during DNA isolation. According to the second method, 60-75% of the MSCs had attached by 6 h (FIG. 4(f)). Regardless of the method used, CD264⁺ and CD264⁻ MSCs had similar attachment efficiencies. After 6 h of mixing, the seeded granules were bound together with mouse thrombin and fibrinogen into a larger 3D construct (<1 cm in diameter) for efficient implantation (FIGS. 4(g-h)).

In Vivo Survival Kinetics

Bioluminescence imaging indicates that ectopic implants of aging CD264⁺ MSCs have similar survival kinetics to matched CD264⁻ MSCs from the same culture (FIG. 5). Representative image sequences and corresponding temporal profiles show intra-mouse comparisons of bioluminescence from matched pairs of CD264⁺ and CD264⁻ implants (FIGS. 5(a-c)). These whole-animal images demonstrate the MSCs remained at the site of implantation.

CD264⁺ and CD264⁻ implants from the same donor had comparable survival kinetics according to the parameters analyzed: rate of BLI signal decay, percent survival calculated from BLI signals during week 1 and 4, and signal half-life (FIGS. 5(d-f)). For each implant, we calculated the following survival metrics: rate of BLI signal decay (data not shown), Week 4 to Week 1 signal ratio (data not shown), and signal half-life (FIGS. 5(d-f)).

More specifically, the decay rate characterizes the exponential decrease in luminescence over time, and the Week 4 to Week 1 signal ratio estimates the percent of hBM-MSCs that survived after 1 month (data not shown). Signal half-life was determined for each sample to allow for intuitive interpretation of survival data and to facilitate meta-analysis across published in vivo MSC survival studies (FIG. 5(d-f)). For each of these parameters, CD264⁺ and CD264⁻ implants from the same donor had comparable survival kinetics. All survival metrics were independent of the colony-forming efficiency of the hBM-MSCs that were implanted, as the slope remains relatively flat over varying colony-forming efficiency.

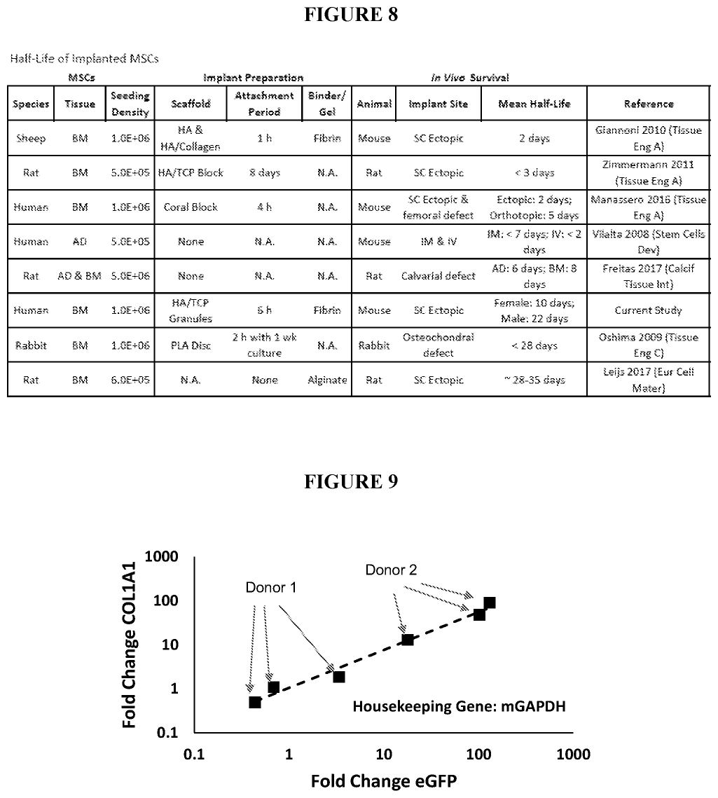

Perhaps most surprisingly, the mean half-lives in this example were among the highest values in the literature (FIG. 8). This surprising result indicates that the robust quality of our implant preparations, as well as the fact the CD264⁺ is not an indicator of in vivo survival despite its positive correlation with cell senescence.

In contrast, there was significant donor-to-donor variation in the in vivo survival kinetics of MSC implants. MSC implants from the donor 2 survived longer with >2-fold difference in mean values of the kinetics parameters between the two donors (p<0.01, n=12 replicates per donor, FIGS. 5 (d-f)). For example, the mean half-life of MSC implants from donor 2 was >20 days (FIG. 5(d)) vs. ˜10 days for implants from donor 1.

Different NG2 Expression Profiles in Donors

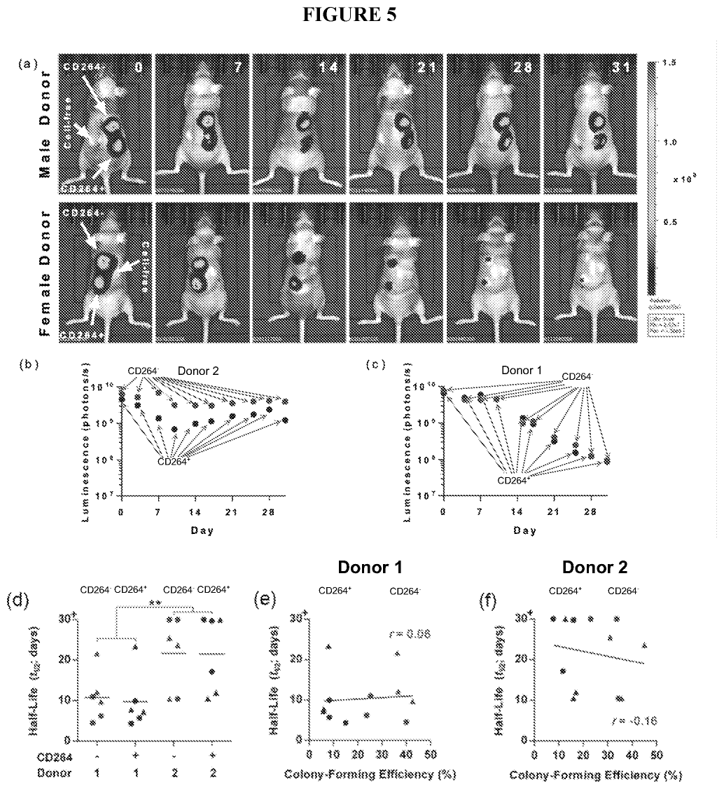

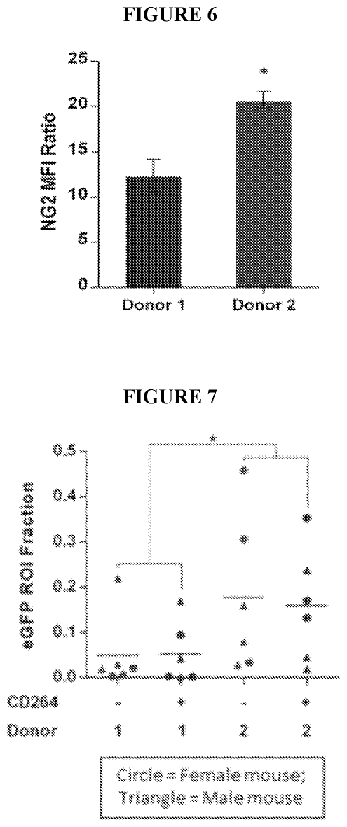

As a comparison, flow cytometric analysis revealed increased NG2 surface expression for high-survival hBM-MSCs from donor 2 relative to donor 1 hBM-MSCs (FIG. 6). The NG2 MFI ratio for donor 2 MSCs was on average >1.5 times the value for donor 1 MSCs (p<0.05, n=3, FIG. 6). These results suggest a link between NG2 surface expression and in vivo survival of MSCs. A 50% difference in NG2 MFI ratio and the 2-fold difference in in vivo survival indicates that NG2 expression may be a good candidate for selecting MSCs that have a higher in vivo survival rate.

Excised Implant

31 days after implant, the mice were sacrificed and the implants were excised to determine the hBM-MSC content thereof. The area fraction occupied by eGFP-positive cells in tissue sections (ROI fraction) was strongly correlated with the percent survival of implanted hBM-MSCs. It is found that the mean eGFP ROI fraction was 3× greater for the hBM-MSCs from donor 2 as compared with donor 1 (FIG. 7). Consistent with the in vivo BLI data, for a given donor, there was no significant difference in eGFP content between aging CD264⁺ and control CD264⁻ implants.

Differentiation Potential

FIG. 9 shows mRNA expression of eGFP and human collagen 1A1 in ectopic implants of eGFP+ human MSCs excised from NIH III mice one month after surgery. Fold change in expression was evaluated with qPCR relative to mouse GAPDH. MSC implants with greater fold change for eGFP had a higher survival half-life. mRNA expression of eGFP and the osteogenesis marker collagen 1A1 were correlated (rₛ>0.95, n=6, FIG. 9). FIG. 9 shows the feasibility of exploiting MSC biological variability to examine the relationship between MSC survival and bone formation.

Specifically, the three upper-right datapoints came from MSC implants obtained from donor 2, whereas the three lower-left datapoints came from MSC implants obtained from donor 1. As discussed above regarding FIG. 6, MSCs obtained from donor 2 have higher NG2 expression than donor 1. The correlation in FIG. 9 indicates that MSCs having higher NG2 expression have a longer in vivo half-life, and also higher differentiation potential.

Taken together, the similarity in the in vivo survival of CD264⁺ and CD264⁻ implants in FIG. 5 is supported by analogous findings on the in vitro single-cell survival of CD264-sorted populations in FIG. 3. These data indicate matched CD264⁺ and CD264⁻ BM-MSCs from the same culture have comparable in vitro and in vivo survival, which is independent of colony-forming efficiency.

In summary, while CD264⁺ has been reported as a marker for MSC senescence, the actual in vivo survival for CD264⁺ and CD264⁻ MSCs shows no significant difference. On the other hand, not only is NG2 expression level an indicator of in vitro MSC proliferation and trilineage potential, its expression is also positively correlated with in vivo MSC survival and differentiation potential, which is important for MSC therapies. Therefore, NG2 is a good indicator for selecting MSCs with both long in vivo halflives and good differentiation potential for MSC therapies.

### Prophetic Example 1: In Vivo MSC Survival of NG2-High/NG2-Low

Human bone marrow MSCs will be collected from healthy donors at passage P3-P4, which is in the range of passage numbers for MSCs in clinical trial. The accepted criteria for human MSCs include: plastic-adherence, potency and immunophenotype. Only MSCs passing the criteria will be further cultured, and these properties will be reevaluated in MSC subsets and genetically modified MSCs.

The in vivo MSC survival will be assessed by bioluminescence imaging with a well-established humanized mouse model of ectopic implant survival. Briefly, human MSCs will be transduced with a lentivirus to express GFP and a luciferase. Implant (˜1 cm diameter) will consists of a fibrin gel containing 106 MSCs attached to 40 mg hydroxyapatite/β-tricalcium phosphate granules (˜1 mm diameter, Medtronic). Attachment of MSCs to the scaffold will be monitored by measuring DNA content extracted from the granules and verified by fluorescence microscopy to detect GFP expression by the transduced MSCs. The granules have a similar mineral composition to that of bone and are used as a bone void filler in oral and maxillofacial surgery. The MSC construct will be implanted subcutaneously on the dorsum of NIH III mice (Charles River Laboratories), which is a standard immunodeficient breed for xenoimplantation of human MSCs. Upon injecting the mice with luciferin, the bioluminescent signal from the implants will be quantified using the IVIS Lumina XRMS In Vivo Imaging System (PerkinElmer). With whole body scans, we verified that MSCs attached to the ceramic granules remain at the implant site. Also, we verified that the bioluminescent signal intensity correlates to the number of implanted GFP-positive MSCs.

Survival metrics: Using the survival model described above, each mouse will be implanted with a pair of NG2^{HI} and NG2^{LO} MSCs and an MSC-free control. We will inject the implanted mice with luciferin and measure the maximum radiance signal emitted by each implant every 3-5 days for a month. We will quantify the half-life of implanted MSCs (endpoint) defined as the (ln 2)/(radiance decay rate). The half-life is calculated using all the radiance data over 31 days and is preferred over the ratio of final-to-initial radiance (alternative), which is prone to larger error. We will validate our results with histological analysis of the area fraction of GFP⁺ MSCs within the implant after excision on day 31. Statistical analysis: Data acquisition and analysis will be blinded wherever possible. Differences in survival half-life between intra-mouse pairs of NG2^{HI} and NG2^{LO} MSCs will be analyzed with a mixed-effects ANOVA model to account for biological variation. A sample size n=7 donor pairs/donor sex will be required to detect differences among the groups based on 80% power, α=0.05, 2-fold difference in half-life and intra-mouse error of 25%.

It is expected to show that MSCs with high expression of NG2 will have significantly longer in vivo halflives as compared to MSCs having low expression of NG2.

### Prophetic Example 2: Modelling In Vivo Survival Prediction Based on In Vitro Data

Predicting in vivo MSC survival is practical in improving MSC therapies. To evaluate the ability of in vitro viability assays to predict in vivo MSC survival, modelling the association between in vivo MSC half-life and in vitro viability endpoints under ischemic stress by nutrient deprivation is proposed.

MSCs will be from randomly selected female and male donors. We will include any sorted MSC groups that exhibit a significant difference in implant survival.

In vivo survival assay: the MSC implant half-life is measured as discussed above.

Single cell survival assay: Single cells will be generated by limiting dilution into 96-well plates and detected by fluorescence microscopy. Endpoint is the percentage of single cells that survive after 7 days as measured by cell attachment.

Nutrient-deprivation assay: Constructs of luciferase-expressing MSCs will be prepared and attached to scaffold granules and encapsulated in a fibrin gel as described above. To mimic ischemia, MSC constructs will be maintained in hypoxic conditions in serum- and glucose-free medium. The O₂ level in the in vitro construct will be measured with a needle microsensor (PreSens Precision Sensing) and will be controlled with an O₂/N₂/CO₂ incubator (Thermo Fisher) to mimic the O₂ level in the in vivo implant as described above. Under these conditions, it is reported that MSC viability declines steadily. Endpoint is the half-life of the bioluminescence from in vitro MSC constructs, which will be measured daily with our PerkinElmer Imaging System.

It is expected that a statistically significant association between in vitro and in vivo survival metrics for one or both in vitro assays as suggested by FIG. 3 for the single cell survival assay.

The following methods were used in this disclosure.

MSC Cultures

Primary MSCs were isolated from iliac crest bone marrow aspirate from healthy adult volunteers with approval of the Tulane Institutional Review Board. Plastic-adherent MSCs prior to expansion were designated as passage 0 (P0). Donor MSC cultures employed in this study satisfy the criteria established by the International Society for Cellular Therapy for defining human MSCs based on plastic-adherence, immunophenotype and differentiation (Dominici et al., 2006). Unless otherwise noted, all cell culture supplies were obtained from Thermo Fisher Scientific (Waltham, Mass., USA). MSCs were routinely cultured in T-flasks using complete culture medium with antibiotics (CCMA): α-MEM with 2 mM L-glutamine supplemented with an additional 2 mM L-glutamine, 100 U/ml penicillin, 100 μg/ml streptomycin, and 20% fetal bovine serum (FBS) (Sekiya et al., 2002). Cultures were inoculated at ≥100 cells/cm² and maintained at 37° C. and 5% CO₂ in a humidified incubator. Medium was completely exchanged every 3-4 days. At 50% confluence, cultures were subcultured using 0.25% trypsin/1 mM EDTA.

Other Cultures

COLO205 (ATCC CCL-222, Manassas, Va., USA) and G361 (ATCC CRL-1424) human cell lines were used for positive controls for cell surface expression of CD264 and NG2, respectively (data not shown). These cells were cultured according to supplier's instructions.

Lentiviral Transduction of MSCs

MSCs were transduced using either copGFP Lentiviral Particles (Santa Cruz Biotechnoloy, Dallas, Tex., USA) to express a bright GFP variant (GFP MSCs) or with RediFect Red Fluc-GFP Lentiviral Particles (PerkinElemer, Waltham, Mass., USA) to express red-shifted Luciola Italica luciferase fused by a T2A self-cleaving linker peptide to enhanced GFP (GFP-FLuc MSCs). P2 MSC cultures were inoculated at 1000 cells/cm², and CCMA was replaced 24 h later with transduction medium: 100 μg/ml protamine sulfate (Sigma Aldrich, St. Louis, Mo., USA) in complete culture media containing no antibiotics (CCM). Medium volume was half of that for routine cultivation to promote transduction. Cultures were infected at a MOI of 20-25 and gently rocked a few times to evenly distribute viral particles over the cells (Lin et al., 2012). Spent medium was replaced with fresh transduction medium after 24 h, and a second dose of viral particles at the same MOI was added. Medium was replaced the following day with fresh CCM at standard volume. After 3 days, GFP-positive cells were collected by fluorescence-activated cell sorting (FACS) and cultured in CCMA until cryopreserved at passage 3.

Flow Cytometry

MSC cultures were amplified to P5 prior to flow cytometric analysis and FACS. Antibodies to detect human CD264 (PE-conjugated, FAB633P) and NG2 (APC-conjugated, FAB2585A) were obtained from R&D Systems (Minneapolis, Minn., USA). Antibodies to detect standard MSC markers were acquired as previously described (Madsen et al., 2017). Following gentle trypsinization and deactivation with CCMA, MSCs were resuspended in PBS at 0.5-1×10⁷ cells/ml. Cell suspensions were incubated with antibody at saturating conditions for 30 min in the dark and on ice. Labeled cells were washed with 1×PBS and 1×4% FBS in PBS, and then resuspended at 2.5×10⁶ cells/ml in chilled 4% FBS in PBS for analysis and sorting.

Flow cytometry was performed with a BD FACSAria Fusion flow cytometer equipped with FACSDiva software (version 8.0.1, BD Biosciences, Franklin Lakes, N.J., USA). Transduced MSCs were analyzed and sorted in tandem with matched isotype and mock-infected controls. Spectral overlap was corrected with multicolor compensation. Samples were gated to eliminate cellular debris and exclude doublets. MSCs were labeled with Fixable Viability Stain 780 (BD Biosciences) to assess viability, which was ≥90%. CD264− and CD264+ populations were sorted by capturing in purity mode MSCs with the bottom and top 10% of PE fluorescence, respectively. Aliquots of sorted cells were reanalyzed for PE fluorescence to validate sort purity. MSCs were sorted into chilled CCMA and then allowed to recover in T-flasks containing CCMA for 36 h prior to further experimentation.

Post hoc flow cytometric analysis was done with Kaluza software (version 1.3, Beckman Coulter, Brea, Calif., USA). MSCs with fluorescence greater than the 99^{th} percentile of the fluorescence distribution for the isotype control were designated positive for antigen expression. Mean fluorescent intensity (MFI) ratios were reported as the MFI for the labeled sample relative to that of the isotype control.

Construct Preparation

Each construct was prepared with 40 mg of Mastergraft Mini Granules (15% hydroxyapatite/85% β-tricalcium phosphate, Medtronic, Memphis, Tenn., USA) (aliquoted into 50 ml vented conical tubes (CELLTREAT, Pepperell, Mass., USA). Granules were washed with 1×PBS and 1×CCMA, and then stored in 10 ml of CCMA overnight. After removing the medium, granules were seeded with 1×10⁶ MSCs in 1 ml of prewarmed CCMA. Cells were mixed with the granules at 50-60 rpm for 6 h at 37° C. in a CO₂ incubator. The MSC construct was centrifuged at 1000×g for 8 minutes, and supernatant was removed. Cell attachment was quantified by measuring (1) cells remaining in solution and (2) DNA content on the granules using the PureLink Genomic DNA Mini Kit (Thermo Fisher Scientific). To bind granules together, 15 μl of mouse fibrinogen (3.2 mg/ml in PBS, Oxford Biomedical Research, Rochester Hills, Mich.) and 15 μl of mouse thrombin (25 U/ml in 2% CaCl₂, Oxford Biomedical Research) were added to each construct and allowed to coagulate for 1 min in a CO₂ incubator (Mankani et al., 2008). After 1 ml of fresh CCMA was added to each tube, the construct was implanted.

In Vivo Survival Assay

Two- to four-month-old male and female NIH-III nude homozygous mice (Charles River Laboratories, Wilmington, Mass., USA) were implanted with MSC constructs with approval of Tulane's Institutional Animal Care and Use Committee. The animals were fed defined Purina LabDiet 5V5R (St. Louis, Mo., USA) starting 2 weeks prior to surgery and throughout the assay. Mice were anesthetized using isofluorane (MWI Animal Health, Boise, Id., USA) and administered 5 mg/kg Meloxicam (MWI Animal Health) subcutaneously prior to surgery. Each mouse was implanted with 3 MSC constructs: (1) CD264⁻, (2) CD264⁺, and (3) a cell-free control. Small incisions (1-2 cm) were made on the dorsal skin surface, and a subcutaneous pocket was created by blunt dissection. A single construct was inserted into each pocket. Incisions were closed with simple interrupted sutures and covered with Vetbond Tissue Adhesive (3M, Maplewood, Minn., USA). The mice were examined with bioluminescence imaging over 31 days and then humanely sacrificed using CO₂ asphyxiation followed by cervical dislocation.

In Vitro Survival Assays

MSCs were stained with 10 μM CellTracker Green (Thermo Fisher Scientific) and plated by limited dilution into 96-well plates. Wells containing a single cell were detected by fluorescence microscopy. Cells were restained with CellTracker Green after 3 days and with crystal violet (Sigma Aldrich) after 1 week to identify single cells that survived and formed colonies (≥10 cells). In vitro survival of MSC constructs was monitored with bioluminescence imaging. Constructs were cultured in 50 ml vented conical tubes with constant mixing at 50-60 RPM and complete medium exchange every 2-3 days. Every 2 weeks for 2 months, the constructs were transferred to 24-well plates containing CCMA for bioluminescence imaging.

Bioluminescence Imaging

Bioluminescence of cell cultures and implants was measured using an IVIS Lumina XRMS In Vivo Imaging System (PerkinElmer) with Living Image software (version 4.4, PerkinElmer). For in vivo imaging, mice were sedated with isofluorane and 100 μl Xenolight D-Luciferin (30 mg/ml, PerkinElmer) was administered subcutaneously adjacent to each implant. For in vitro imaging, MSC constructs were exposed to 300 μg/ml D-luciferin in CCMA. Bioluminescence was acquired every 5 min after luciferin addition using the automatic exposure settings until the bioluminescent signal decreased. Maximum radiance in the region of interest around each construct was measured every 3-4 days for 31 days and background corrected. When grouped together, representative bioluminescent images were placed on an identical radiance color scale. Radiance data were natural log-transformed, and a linear regression was performed. The slope of the regression line corresponds to the rate of radiance decay. Signal half-life (t_{1/2}) was calculated from the decay rate (λ) using the following formula:

$${t}_{1 / 2} = \frac{ln 2}{\vert λ \vert} .$$

Other Assays

Colony-forming efficiency was evaluated according to Barrilleaux et al. (2009). MSCs were plated at a clonogenic level of 100±10 cells in a 10 cm cell culture dish with 15 ml CCMA. Samples were cultured undisturbed for 14 days and then stained with crystal violet to detect cell colonies (≥50 cells). Senescence-associated β-galactosidase (SA β-Gal) activity at pH 6.0 was assessed in subconfluent MSC cultures using Senescence Cells Histochemical Staining Kit (Sigma Aldrich). MSCs stained at pH 5.0 served as a positive β-Gal control. Osteo-, adipo-, and chondrogenesis were induced in MSCs and evaluated after 21 days of differentiation. Alizarin Red S (Sigma Aldrich) detected calcified extracellular matrix in osteogenic samples, AdipoRed (Lonza, Walkersville, Md., USA) stained lipid droplets in adipogenic cells, and Alcian Blue (Sigma Aldrich) identified matrix deposition of sulfated glycosaminoglycans during chondrogenesis.

The following references are incorporated by reference in their entirety for all purposes.
Chang et al., Clearance of senescent cells by ABT263 rejuvenates aged hematopoietic stem cells in mice. Nature Medicine (2016), vol. 22 (1), 78-86.
Ehrhardt et al., TRAIL induced survival and proliferation in cancer cells resistant towards TRAIL-induced apoptosis mediated by NF-κB. Oncogene (2003), 22(25), 3842-3852. Available at: doi.org/10.1038/sj.onc.1206520
Lalaoui et al., TRAIL-R4 promotes tumor growth and resistance to apoptosis in cervical carcinoma HeLa cells through AKT. PLOS One (2011), 6(5), Available at: doi.org/10.1371/journal.pone.0019679. e19679
López-Gómez et al., TRAIL and TRAIL receptors splice variants during long-term interferon β treatment of patients with multiple sclerosis: Evaluation as biomarkers for therapeutic response. Journal of Neurology, Neurosurgery and Psychiatry, (2016) 87(2), 130-137. Available at: doi.org/10.1136/jnnp-2014-309932
Madsen et al., Decoy TRAIL receptor CD264: A cell surface marker of cellular aging for human bone marrow-derived mesenchymal stem cells. Stem Cell Research & Therapy (2017), 8(1), 201. Available at: doi.org/10.1186/s13287-017-0649-4
Manassero et al., Comparison of survival and osteogenic ability of human mesenchymal stem cells in orthotopic and ectopic sites in mice. Tissue Engineering (2016), Part A, 22(5-6), 534-544. Available at: doi.org/10.1089/ten.TEA.2015.0346
Panneerselvam et al., Role of decoy molecules in neuronal ischemic preconditioning. Life Sciences (2011), 88(15-16), 670-674. Available at: doi.org/10.1016/j.lfs.2011.02.004
Robey et al., Bone marrow stromal cell assays: In vitro and in vivo. Methods in Molecular Biology (2014), 1130(6), 279-293. Available at: doi.org/10.1007/978-1-62703-989-5_21
Russell et al., Cell-surface expression of neuron-glial antigen 2 (NG2) and melanoma cell adhesion molecule (CD146) in heterogeneous cultures of marrow-derived mesenchymal stem cells. Tissue Engineering, Part A (2013), 19(19-20), 2253-2266. Available at: doi.org/10.1089/ten.TEA.2012.0649
Zhu et al., Effects of estrogen on stress-induced premature senescence of vascular smooth muscle cells: A novel mechanism for the “time window theory” of menopausal hormone therapy. Atherosclerosis (2011), 215(2), 294-300. Available at: doi.org/10.1016/j.atherosclerosis.2010.12.025

What is claimed is:

1. A method of making a scaffold having mesenchymal stem cells (MSCs) attached thereon, comprising the steps of:
a) selecting, from a heterogeneous group of MSCs, MSCs having high expression of neuron-glial antigen 2 (NG2);
b) expanding the MSCs selected in step a); and
c) attaching the MSCs expanded in step b) to a scaffold.

2. The method of claim 1, wherein in step a) the selection is based on NG2 expression level in the heterogeneous group of MSCs, and wherein only the MSCs having top 30% expression level of NG2 are selected.

3. The method of claim 2, wherein only the MSCs having top 15% expression level of NG2 are selected.

4. The method of claim 2, wherein only the MSCs having top 10% expression level of NG2 are selected.

5. The method of claim 1, wherein after step b) further comprising:
b-2) removing MSCs having CD264⁺.

6. The method of claim 5, wherein in step c) less than 10% of the expanded MSCs are CD264⁺.

7. The method of claim 1, wherein after step b) further comprising:
b-3) enriching MSCs having CD264⁺.

8. The method of claim 7, further comprising:
e) removing or killing the CD264⁺ MSCs from said implant after a predetermined period of time.

9. The method of claim 0, wherein the scaffold is made of a material selected from the group consisting of hydroxyapatite-tricalcium phosphate (HA-TCP) granules, hydrogel, PLGA, collagen gel, spongastan, matrigel, fibronectin, and combinations thereof.

10. The method of claim 9, wherein the scaffold is made of HA/TCP granules.

11. A composition, comprising a scaffold having mesenchymal stem cells (MSCs) attached thereon, wherein at least 70% of the MSCs have high expression of NG2 and less than 30% of the MSCs are CD264⁺.

12. The composition of claim 11, wherein wherein the scaffold is made of a material selected from the group consisting of hydroxyapatite/tricalcium phosphate (HA/TCP) granules, hydrogel, PLGA, collagen gel, spongastan, matrigel, fibronectin, and combinations thereof.

13. The composition of claim 11, wherein less than 10% of the MSCs are CD264⁺.

14. A method of implanting mesenchymal stem cells (MSCs) in a mammal, comprising the steps of:
a) selecting, from a heterogeneous group of MSCs, MSCs having top 30% expression level of neuron-glial antigen 2 (NG2);
b) expanding the MSCs selected in step a), wherein the expanded MSCs having CD264⁺ are removed;
c) attaching the MSCs expanded in step b) to a scaffold; and
d) implanting the scaffold with the MSCs in a mammal.

15. The method of claim 14, wherein in step c) less than 10% of the expanded MSCs are CD264+.

16. The method of claim 14, wherein in step a) only MSCs having top 10% expression level of NG2 are selected.

17. A method of implanting mesenchymal stem cells (MSCs) in a mammal, comprising the steps of:
a) selecting, from a heterogeneous group of MSCs, MSCs having top 30% expression level of neuron-glial antigen 2 (NG2);
b) expanding the MSCs selected in step a), wherein the expanded MSCs having CD264⁺ are enriched;
c) attaching the MSCs expanded in step b) to a scaffold; and
d) implanting the scaffold with the MSCs in a mammal.

18. The method of claim 17, wherein in step c) at least 40% of the expanded MSCs are CD264+.

19. The method of claim 17, wherein in step a) only MSCs having top 10% expression level of NG2 are selected.

20. A method of making a scaffold having mesenchymal stem cells (MSCs) attached thereon, comprising the steps of:
a) selecting, from a heterogeneous group of MSCs, MSCs that do not express CD264;
b) expanding the MSCs selected in step a), wherein the expanded MSCs having the bottom 50% expression level of NG2 are removed; and
c) attaching the MSCs expanded in step b) to a scaffold.

21. The method of claim 20, wherein in step b) the expanded MSCs having the bottom 70% expression level of NG2 are removed.
